(19) United States
(12) Patent Application Publication
(10) Pub. No.: US 2024/0325247 A1
(43) Pub. Date: Oct. 3, 2024

(54) MEDICINE ADMINISTERING SYSTEM

(71) Applicant: William Thomas, Moline, IL (US)

(72) Inventor: William Thomas, Moline, IL (US)

(21) Appl. No.: 18/130,421

(22) Filed: Apr. 3, 2023

(51) Int. Cl. A61J 1/20; A61M 5/178; A61M 5/31

(52) U.S. Cl. CPC A61J 1/2096; A61M 5/1782; A61M 5/31

(57) ABSTRACT

A medicine administering system for facilitating administering medicine to the syringe using a downward flow of the medicine from the vial includes a vial containing medicine and a syringe. A base includes a front surface, a back surface, a top edge, a bottom edge, and a pair of lateral edges. A stand is coupled to the base and supports the base in an inclined position relative to a ground surface. A syringe mount is coupled to the base and the syringe is removably mountable to the syringe mount. A vial mount is coupled to the base and the vial is removably mountable to the vial mount. The vial mount is positioned proximate to the top edge, wherein the vial mount is positioned above the syringe mount such that medicine from the vial flows downwardly into the syringe when the syringe receives medicine from the vial.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to syringe and vial systems and more particularly pertains to a new syringe and vial system for facilitating administering medicine to the syringe using a downward flow of the medicine from the vial.

### (2) Description of Related Art Including Information Disclosed Under 37 CFR 1.97 and 1.98

The prior art relates to syringe and vial systems and includes a variety of systems facilitating administering medicine from the vial to the syringe. Furthermore, a variety of systems have an optical magnification glass used to enlarge the readings on the syringe. Known prior art does not include a system being positioned in an inclined position such that a downward flow of the medicine from the vial facilitates administering medicine to the syringe.

### (h) BRIEF SUMMARY OF THE INVENTION

An embodiment of the disclosure meets the needs presented above by generally comprising a vial containing medicine, and a syringe is configured for withdrawing medicine from the vial. The syringe includes a barrel enclosing an interior space. A plurality of graduations is positioned on the barrel. A base includes a front surface, a back surface, a top edge, a bottom edge, and a pair of lateral edges. A stand is coupled to the base and is configured to support the base in an inclined position relative to a ground surface, wherein the inclined position is configured to be defined by the bottom edge abutting the ground surface and the back surface and the ground surface forming an acute angle facing outwardly from the bottom edge. A syringe mount is coupled to the base and the syringe is removably mountable to the syringe mount. A vial mount is coupled to the base and the vial is removably mountable to the vial mount. The vial mount is positioned proximate to the top edge, wherein the vial mount is positioned above the syringe mount such that medicine from the vial flows downwardly into the syringe when the syringe receives medicine from the vial. A magnifier is coupled to the base and is positionable relative to the base and the barrel of the syringe to facilitate viewing of the graduations on the barrel of the syringe.

Another embodiment of the disclosure comprises a method including the steps of standing a base in an inclined position relative to a ground surface. The base has a vial mount and a syringe mount attached thereto. The vial mount is positioned above the syringe mount. A vial mounts to the vial mount and a cap of the vial mount is positioned facing downwardly. A syringe mounts to the syringe mount and a needle of the syringe is positioned facing upwardly. The needle of the syringe inserts through the cap into the vial. A plunger of the syringe pulls to suction medicine from the vial into a barrel of the syringe. The needle removes from the cap of the vial thereafter. The syringe removes from the syringe mount and the vial removes from the vial mount.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top isometric view of a medicine administering system according to an embodiment of the disclosure.

FIG. 2 is a front view of an embodiment of the disclosure.

FIG. 3 is a front view of an embodiment of the disclosure.

FIG. 4 is a cross-sectional view of an embodiment of the disclosure taken along Line 4-4 of FIG. 2.

FIG. 5 is a cross-sectional view of an embodiment of the disclosure taken along Line 5-5 of FIG. 3.

FIG. 6 is a side exploded view of an embodiment of the disclosure.

FIG. 7 is an in-use view of an embodiment of the disclosure.

### (j) DETAILED DESCRIPTION OF THE INVENTION

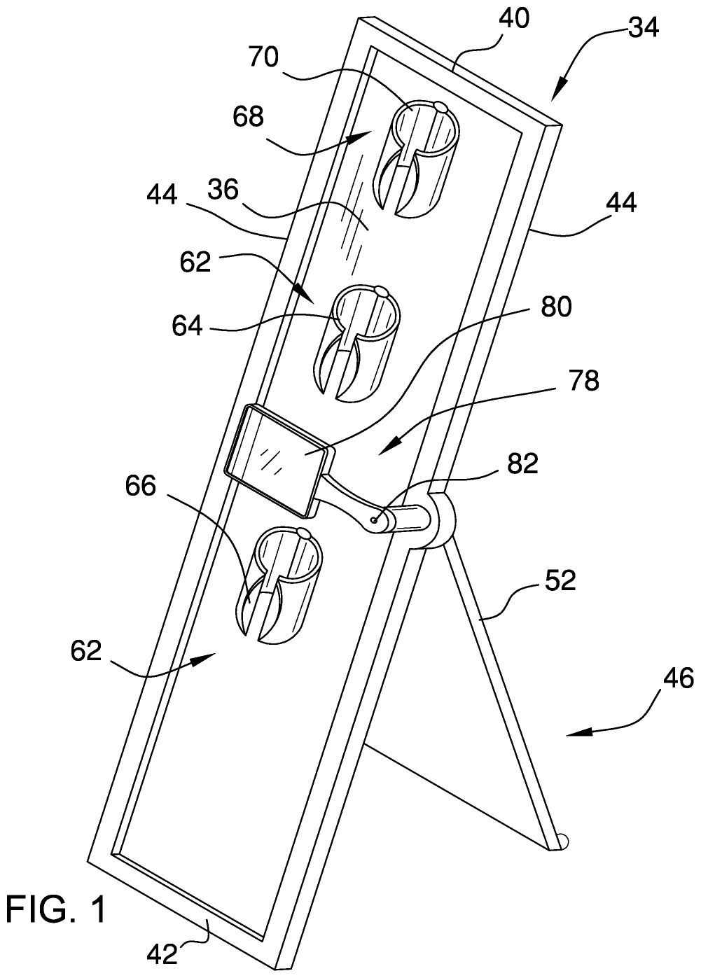

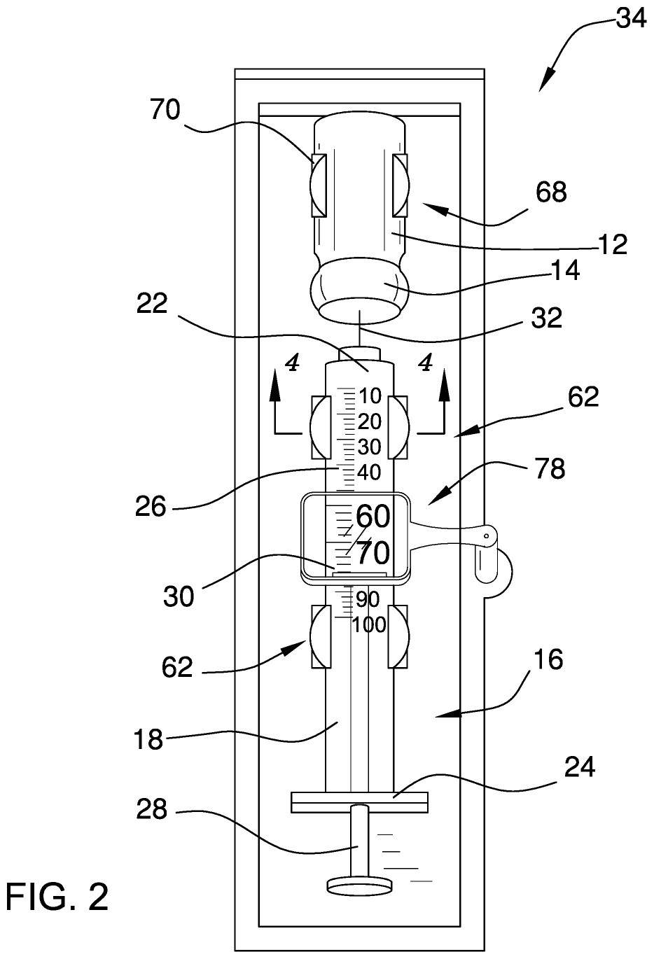

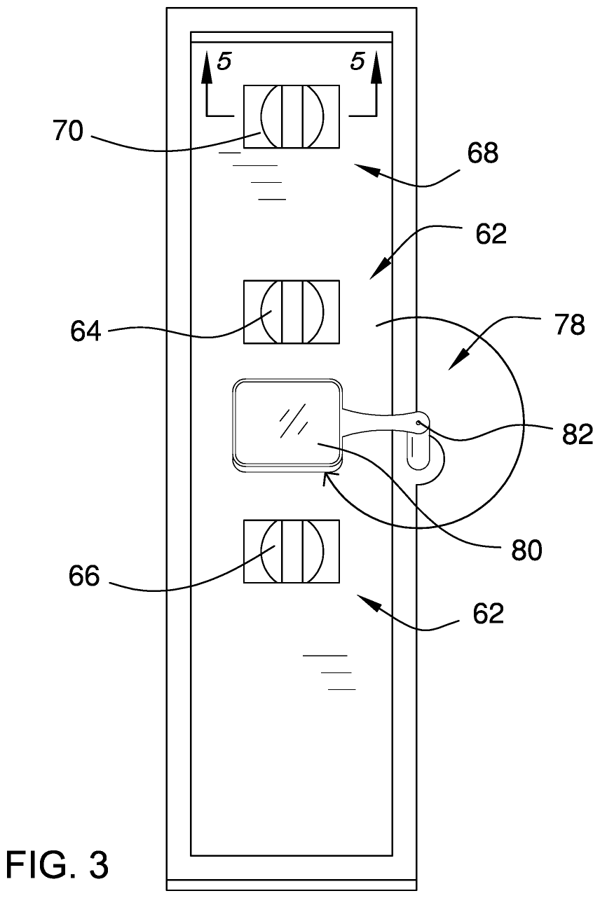

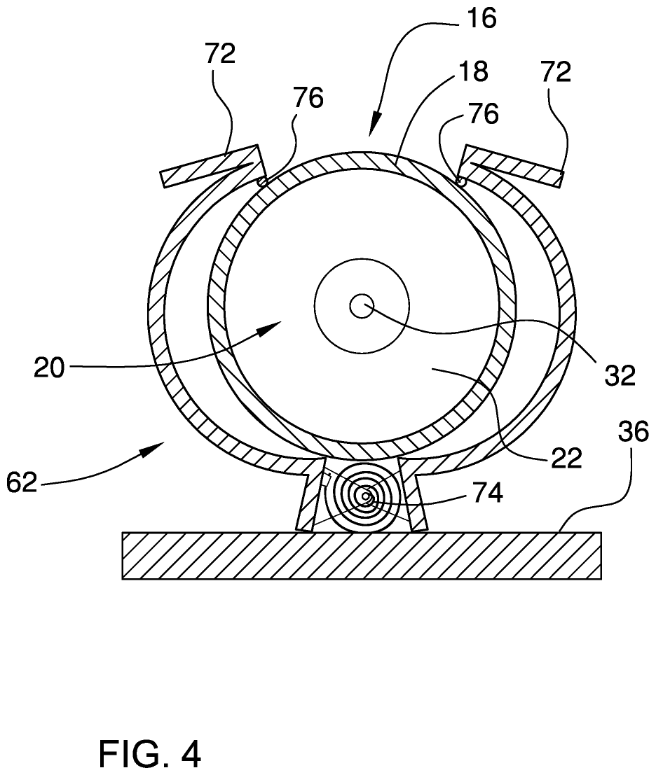

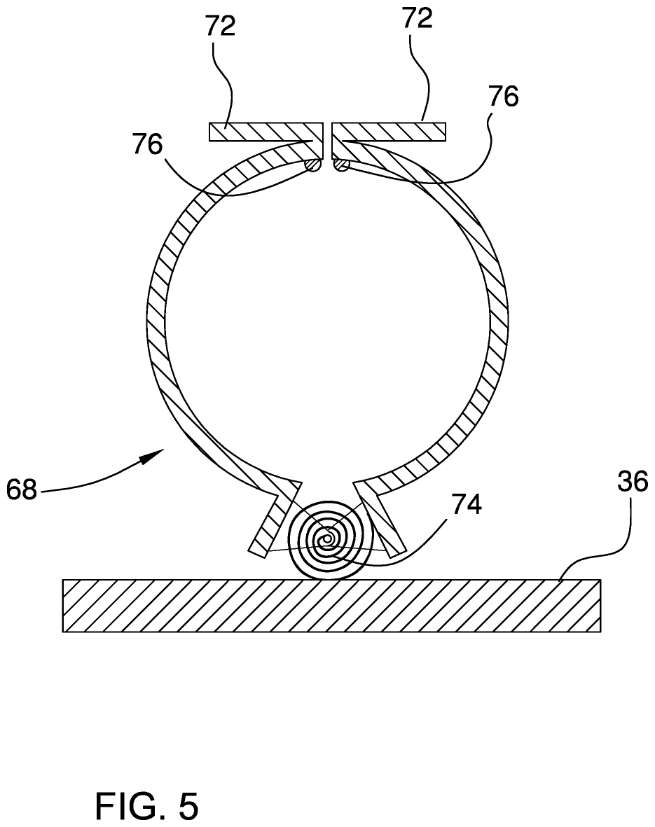

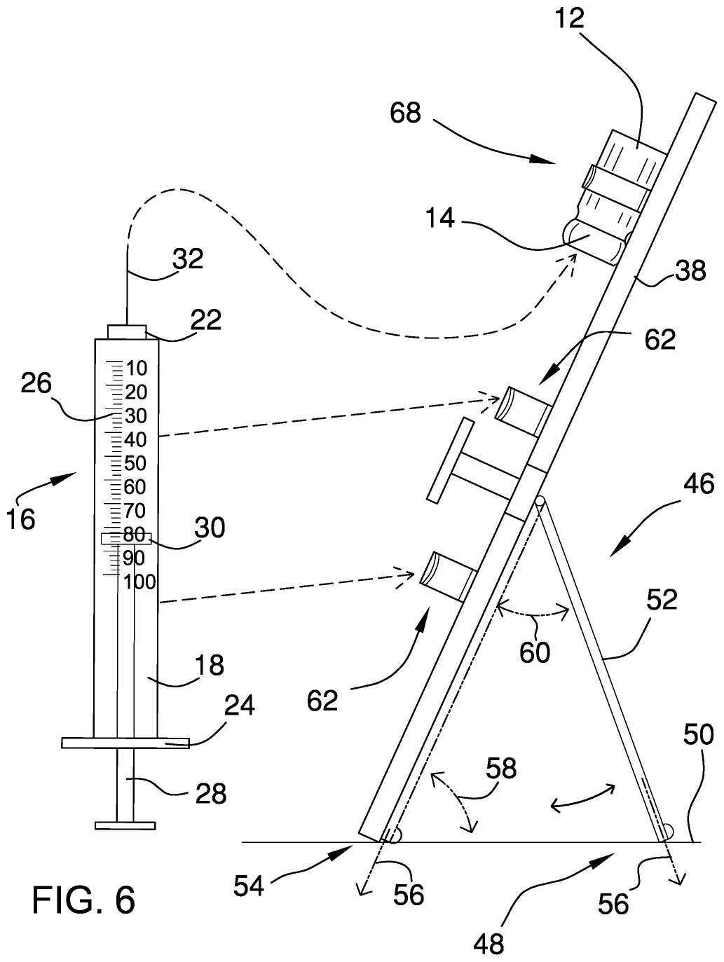

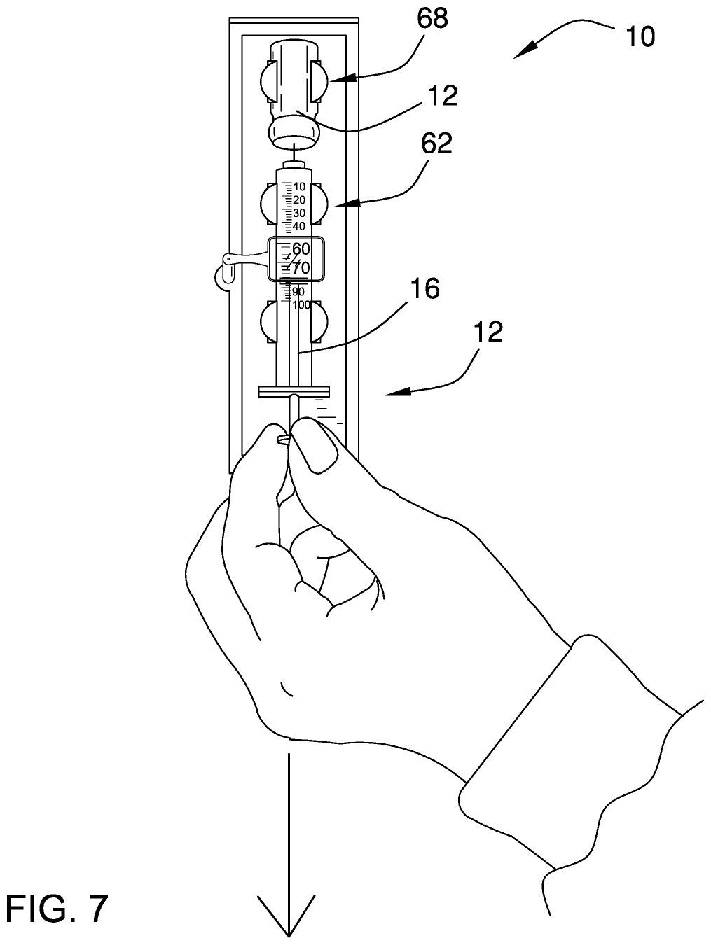

With reference now to the drawings, and in particular to FIGS. 1 through 7 thereof, a new syringe and vial system embodying the principles and concepts of an embodiment of the disclosure and generally designated by the reference numeral 10 will be described.

As best illustrated in FIGS. 1 through 7, the medicine administering system 10 generally comprises a vial 12 containing medicine. A cap 14 is removably positioned on the vial 12 and seals medicine within the vial 12. A syringe 16 is configured for withdrawing medicine from the vial 12. The syringe 16 includes a barrel 18 enclosing an interior space 20. The barrel 18 has a first end 22 and a second end 24. The first end 22 and the second end 24 each are open. A plurality of graduations 26 is positioned on the barrel 18. The plurality of graduations 26 measure a volume of the interior space 20 from one of the first 22 or second 24 ends to a respective graduation 26.

A plunger 28 is inserted through the second end 24 of the barrel 18 into the interior space 20. The plunger 28 is configured to suction medicine into the interior space 20 or expel medicine from the interior space 20. A tip of the plunger 28 has a seal positioned thereon to define a plunger piston 30. The plunger piston 30 is slidably positioned within the interior space 20. The plunger 28 suctions medicine when the plunger piston 30 moves from the first end 22 to the second end 24 within the interior space 20 and expels medicine when the plunger piston 30 moves from the second end 24 to the first end 22. A needle 32 is coupled to the first end 22 of the barrel 18 and is removably insertable through the cap 14 into the vial 12 containing medicine.

A base 34 includes a front surface 36, a back surface 38, a top edge 40, a bottom edge 42, and a pair of lateral edges 44. The lateral edges 44 are elongated, and the base 34 has a rectangular configuration. A stand 46 is coupled to the base 34 and is configured to support the base 34 in an inclined position 48 relative to a ground surface 50, wherein the inclined position 48 is configured to be defined by the bottom edge 42 abutting the ground surface 50 and the back surface 38 and the ground surface 50 forming a first acute angle 58 facing outwardly from the bottom edge 42. The stand 46 comprises a leg 52 pivotally attached to the back surface 38 and extending out from the back surface 38. The leg 52 is positioned from a collapse position 54 wherein a longitudinal axis 56 of the leg 52 is positioned parallel to the back surface 38 to the inclined position 48 wherein the longitudinal axis 56 of the leg 52 and the back surface 38 forms a second acute angle 60 facing the ground surface 50.

A syringe mount 62 is coupled to the base 34 and the syringe 16 is removably mountable to the syringe mount 62. The needle 32 of the syringe 16 is positioned facing towards the top edge 40 when the syringe 16 is mounted to the base 34. The syringe mount 62 comprises a first clamp 64 and a second clamp 66 each attached to a center of the front surface 36. A vial mount 68 is coupled to the base 34 and the vial 12 is removably mountable to the vial mount 68. The vial mount 68 is positioned proximate to the top edge 40, wherein the vial mount 68 is positioned above the syringe mount 62 such that medicine from the vial 12 flows downwardly into the syringe 16 when the syringe 16 receives medicine from the vial 12. The cap 14 of the vial 12 is faced towards the bottom edge 42, wherein the cap 14 is positioned facing the needle 32 of the syringe 16 when the vial 12 is mounted to the base 34. The vial mount 68 comprises a third clamp 70 is attached to the front surface 36. The first clamp 64, the second clamp 66, and the third clamp 70 each include a pair of jaws 72 pivotally attached to a spring-loaded hinge 74 that biases each jaw 72 of the pair of jaws 72 to abut each other. The pair of jaws 72 are selectively movable away from each other to receive the barrel 18 of the syringe 16 or the vial 12. Each of the jaws 72 may have a retaining member 76 attached to there and be configured to retrain the syringe 16 or the vial 14 between the pair of jaws 72.

A magnifier 78 is coupled to the base 34. The magnifier 78 is positionable relative to the base 34 and the barrel 18 of the syringe 16 to facilitate viewing of the graduations 26 on the barrel 18 of the syringe 16. The magnifier 78 comprises a magnifying glass 80 pivotally coupled to a pivoting member 82 attached to the front surface 36 of the base 34. The magnifying glass 80 is positionable over the barrel 18 such that the magnifying glass 80 provides an optical enlargement of the barrel 18 therethrough.

In use, the base 34 stands in an inclined position 48 relative to the ground surface 50. The vial 12 mounts to the vial mount 68 and the cap 14 of the vial 12 is positioned facing downwardly. The syringe 16 mounts to the syringe mount 62 and the needle 32 of the syringe 16 is positioned facing upwardly. The needle 32 of the syringe 16 inserts through the cap 14 into the vial 12. The plunger 28 of the syringe 16 is pulled to suction medicine from the vial 12 into the barrel 18 of the syringe 16. The needle 32 removes from the cap 14 of the vial 12 thereafter. The syringe 16 removes from the syringe mount 62 and the vial 12 removes from the vial mount 68.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medicine administering system comprising:
a vial containing medicine;
a syringe being configured for withdrawing medicine from said vial, said syringe including:
a barrel enclosing an interior space;
a plurality of graduations being positioned on said barrel;
a base, said base including a front surface, a back surface, a top edge, a bottom edge, and a pair of lateral edges;
a stand being coupled to said base and being configured to support said base in an inclined position relative to a ground surface, wherein said inclined position is configured to be defined by said bottom edge abutting the ground surface and said back surface and the ground surface forming a first acute angle facing outwardly from the bottom edge;
a syringe mount being coupled to said base, said syringe being removably mountable to said syringe mount;
a vial mount being coupled to said base, said vial being removably mountable to said vial mount, said vial mount being positioned proximate to said top edge, wherein said vial mount is positioned above said syringe mount such that medicine from said vial flows downwardly into said syringe when said syringe receives medicine from said vial; and
a magnifier being coupled to said base, said magnifier being positionable relative to said base and said barrel of the syringe to facilitate viewing of said graduations on said barrel of said syringe.

2. The medicine administering system of claim 1, further including a cap being removably positioned on said vial, said cap sealing medicine within said vial.

3. The medicine administering system of claim 2, wherein said barrel has a first end and a second end, said first end and said second end each being open.

4. The medicine administering system of claim 3, wherein said plurality of graduations measure a volume of said interior space from one of said first or second ends to a respective graduation.

5. The medicine administering system of claim 4, further including a plunger being inserted through said second end into said interior space, said plunger being configured to suction medicine into said interior space or expel medicine from said interior space, a tip of said plunger having a seal positioned thereon to define a plunger piston, said plunger piston being slidably positioned within said interior space, said plunger suctioning medicine when said plunger piston moves from said first end to said second end within said interior space, said plunger expelling medicine when said plunger piston moves from said second end to said first end.

6. The medicine administering system of claim 5, further including a needle being coupled to said first end of said barrel, said needle being removably insertable through said cap into said vial containing medicine.

7. The medicine administering system of claim 1, wherein said lateral edges are elongated, said base having a rectangular configuration.

8. The medicine administering system of claim 1, wherein said stand comprises a leg being pivotally attached to said back surface and extending out from said back surface, said leg being positioned from a collapse position wherein a longitudinal axis of said leg is positioned parallel to said back surface to the inclined position wherein said longitudinal axis of said leg and said back surface forming a second acute angle facing the ground surface.

9. The medicine administering system of claim 6, wherein said needle of said syringe is positioned facing towards said top edge when said syringe is mounted to said base, said syringe mount comprising a first clamp and a second clamp each attached to a center of said front surface.

10. The medicine administering system of claim 9, wherein said cap of said vial is faced towards said bottom edge, wherein said cap is positioned facing said needle of said syringe when said vial is mounted to said base, said vial mount comprising a third clamp attached to said front surface.

11. The medicine administering system of claim 6, wherein said graduation reading enhancer comprises a magnifying glass being pivotally coupled to said front surface of said base, said magnifying glass being positionable over said barrel such that said magnifying glass provides an optical enlargement of the barrel therethrough.

12. A medicine administering system comprising:
a vial containing medicine;
a cap being removably positioned on said vial, said cap sealing medicine within said vial;
a syringe being configured for withdrawing medicine from said vial, said syringe including:
a barrel enclosing an interior space, said barrel having a first end and a second end, said first end and said second end each being open;
a plurality of graduations being positioned on said barrel, said plurality of graduations measuring a volume of said interior space from one of said first or second ends to a respective graduation;
a plunger being inserted through said second end into said interior space, said plunger being configured to suction medicine into said interior space or expel medicine from said interior space, a tip of said plunger having a seal positioned thereon to define a plunger piston, said plunger piston being slidably positioned within said interior space, said plunger suctioning medicine when said plunger piston moves from said first end to said second end within said interior space, said plunger expelling medicine when said plunger piston moves from said second end to said first end; and
a needle being coupled to said first end of said barrel, said needle being removably insertable through said cap into said vial containing medicine;
a base, said base including a front surface, a back surface, a top edge, a bottom edge, and a pair of lateral edges, said lateral edges being elongated, said base having a rectangular configuration;
a stand being coupled to said base and being configured to support said base in an inclined position relative to a ground surface, wherein said inclined position is configured to be defined by said bottom edge abutting the ground surface and said back surface and the ground surface forming a first acute angle facing outwardly from the bottom edge, said stand comprising a leg being pivotally attached to said back surface and extending out said back surface, said leg being positioned from a collapse position wherein a longitudinal axis of said leg is positioned parallel to said back surface to the inclined position wherein said longitudinal axis of said leg and said back surface forming a second acute angle facing the ground surface;
a syringe mount being coupled to said base, said syringe being removably mountable to said syringe mount, said needle of said syringe being positioned facing towards said top edge when said syringe being mounted to said base, said syringe mount comprising a first clamp and a second clamp each attached to a center of said front surface;
a vial mount being coupled to said base, said vial being removably mountable to said vial mount, said vial mount being positioned proximate to said top edge, wherein said vial mount is positioned above said syringe mount such that medicine from said vial flows downwardly into said syringe when said syringe receives medicine from said vial, said cap of said vial being faced towards said bottom edge, wherein said cap is positioned facing said needle of said syringe when said vial is mounted to said base, said vial mount comprising a third clamp attached to said front surface; and
a magnifier being coupled to said base, said magnifier being positionable relative to said base and said barrel of the syringe to facilitate viewing of said graduations on said barrel of said syringe, said magnifier comprising a magnifying glass being pivotally coupled to said front surface of said base, said magnifying glass being positionable over said barrel such that said magnifying glass provides an optical enlargement of the barrel therethrough.

13. A method for administering medicine to a syringe including the steps of:
standing a base in an inclined position relative to a ground surface, said base having a vial mount and a syringe mount attached thereto, said vial mount being positioned above said syringe mount;
mounting a vial to said vial mount, a cap of said vial mount being positioned facing downwardly;
mounting a syringe to said syringe mount, a needle of said syringe being positioned facing upwardly;
inserting said needle of said syringe through said cap into said vial;
pulling a plunger of said syringe to suction medicine from said vial into a barrel of said syringe;
removing said needle from said cap of said vial;
removing said syringe from said syringe mount; and
removing said vial from said vial mount.
